SEQUENCE LISTING (38 sequences)

SEQ ID NO: 1 - protein, 238 aa, Homo sapiens [sequence omitted]

SEQ ID NO: 2 - protein, 238 aa, Homo sapiens
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 3 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 5 - protein, 430 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 6 - DNA, 1302 bp, Homo sapiens
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggcggccgcggtggcggaggtagtggtggcggaggtagcggtggcggaggttctggtggcggaggttccgaattcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaaaagcttttcgaa

SEQ ID NO: 7 - protein, 436 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQKLFDTG

SEQ ID NO: 8 - DNA, 1326 bp, Homo sapiens
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggcggccgcggtggcggaggtagtggtggcggaggtagcggtggcggaggttctggtggcggaggttccgaattcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaaaagcttttcgataccggtcatcatcaccatcaccat

SEQ ID NO: 9 - protein, 473 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQKLFDTG

SEQ ID NO: 10 - DNA, 1401 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggcggccgcggtggcggaggtagtggtggcggaggtagcggtggcggaggttctggtggcggaggttccgaattcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 11 - protein, 473 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQKLFDTG

SEQ ID NO: 12 - DNA, 1401 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggcggccgcggtggcggaggtagtggtggcggaggtagcggtggcggaggttctggtggcggaggttccgaattcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatcgcaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 13 - DNA, 36 bp, Artificial Sequence
  Leptin Primer Foward
gggaaagctagccaccatgcattggggaaccctgtg

SEQ ID NO: 14 - DNA, 36 bp, Artificial Sequence
  Leptin Primer Reverse
attgattagcggccgccgcacccagggctgaggtcc

SEQ ID NO: 15 - DNA, 21 bp, Artificial Sequence
  GCSF Primer Foward
aagctggctagccaccatggc

SEQ ID NO: 16 - DNA, 29 bp, Artificial Sequence
  GCSF Primer Reverse
aattaatagcggccgccgggctgggcaag

SEQ ID NO: 17 - protein, 443 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQKLFDTG

SEQ ID NO: 18 - protein, 443 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQKLFDTG

SEQ ID NO: 19 - protein, 409 aa, homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 20 - protein, 409 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGRGGGGSGGGGSGGGGSGGGGSEFFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 21 - protein, 256 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 22 - protein, 256 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 23 - DNA, 1290 bp, Artificial Sequence
  fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 24 - protein, 409 aa, Artificial Sequence
  Fusion Protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 25 - DNA, 1290 bp, Artificial Sequence
  fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcggtggcggaggtagtggtggcggaggtagcggtggcggaggttctggtggcggaggttccggtggcggaggtagtttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatcgcaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 26 - protein, 409 aa, Artificial Sequence
  Fusion Protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSGGGGSGGGGSGGGGSGGGGSFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 27 - DNA, 1215 bp, Artificial Sequence
  Fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 28 - protein, 384 aa, Artificial Sequence
  fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 29 - DNA, 1215 bp, Artificial Sequence
  fusion protein
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatcgcaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 30 - protein, 384 aa, Artificial Sequence
  fusion protein
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 31 - DNA, 1401 bp, Artificial Sequence
  Fusion Protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatccaaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 32 - protein, 437 aa, Artificial Sequence
  Fusion Protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 33 - DNA, 1401 bp, Artificial Sequence
  Fusion Protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccggtggcggaggtagtggtggcggaggtagcggtggcggaggttctggtggcggaggttccggtggcggaggtagtttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatcgcaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 34 - protein, 437 aa, Artificial Sequence
  Fusion Protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSGGGGSGGGGSFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 35 - DNA, 1326 bp, Artificial Sequence
  Fusion Protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatccaaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 36 - protein, 412 aa, Artificial Sequence
  Fusion Protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

SEQ ID NO: 37 - DNA, 1326 bp, Artificial Sequence
  Fusion Protein
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatcgcaataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtacgcgtgagatccaaacaacgaaactctggaaattatggcgagttcagtgaggtgctctatgtaacacttcctcagatgagccaa

SEQ ID NO: 38 - protein, 412 aa, Artificial Sequence
  Fusion Protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIAIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQ

(19) United States
(12) Patent Application Publication
(10) Pub. No.: US 2019/0085042 A1
(43) Pub. Date: Mar. 21, 2019

(54) FUSION POLYPEPTIDE COMPRISING THE EXTRACELLULAR BINDING DOMAIN OF GROWTH HORMONE RECEPTOR

(71) Applicant: Asterion Limited, Sheffield (GB)

(72) Inventors: Ian Wilkinson, Sheffield (GB); Richard Ross, Sheffield (GB); Peter Artymiuk, (US)

(73) Assignee: Asterion Limited, Sheffield (GB)

(21) Appl. No.: 15/766,899

(22) PCT Filed: Oct. 18, 2016
(86) PCT No.: PCT/GB2016/053218

§ 371 Date: Apr. 9, 2018

(30) Foreign Application Priority Data
Nov. 13, 2015 (GB) 1520021.5

(51) Int. Cl. C07K 14/535; C07K 14/575; C07K 14/72; C12N 15/62

(52) U.S. Cl. CPC C07K 14/535; C07K 14/5759; C07K 2319/31; C12N 15/62; C07K 14/72

(57) ABSTRACT

The invention relates to fusion polypeptides comprising the extracellular domain of growth hormone linked either directly or indirectly to a polypeptide wherein the polypeptide is not growth hormone.

### CROSS REFERENCE TO RELATED APPLICATIONS

This is the U.S. National Stage of International Application No. PCT/GB2016/053218, filed Oct. 18, 2016, which was published in English under PCT Article 21(2), which in turn claims the benefit of Great Britain Application No. 1520021.5, filed Nov. 13, 2015.

### FIELD OF THE INVENTION

The invention relates to a fusion polypeptide comprising the extracellular binding domain of a growth hormone receptor; pharmaceutical compositions comprising the fusion polypeptide and uses of the fusion polypeptide in the treatment of diseases and conditions that would benefit from administration of the fusion polypeptide.

### BACKGROUND TO THE INVENTION

A problem associated with recombinant proteins and peptides that are used as biopharmaceuticals is serum clearance. Factors that result in the removal of administered proteins from the circulation have two components; renal filtration and proteolysis. Typically, proteins with a molecular weight above 70 kDa are not cleared by glomerular filtration because they are simply too large to be filtered. Certain proteins of small molecular weight are filtered by the glomerulus and are found in the urine. For example, growth hormone [GH] has a molecular weight of 22.1 kDa and the kidney is responsible for clearing up to 60-70% of GH in humans. Other examples of relatively small molecular weight proteins which are filtered by the kidney include leptin, erythropoeitin, and IL-6.

It is known in the art to modified protein biopharmaceuticals to retard serum clearance. For example, Syed et al [Blood, 89, 3243-3252, (1997)] constructed an anti-coagulant fusion protein which fused hirudin with albumin. This fusion protein showed extended plasma half-life whilst maintaining a potent anti-thrombin (anti-coagulant) activity. However a problem associated with this strategy is that hirudin is a foreign protein which is known to provoke a strong immune response. Similarly, blood clotting Factor VII and VIIa has been fused to albumin to extend serum half-life [see WO2007/090584 and Weimer et al Thromb Haemost, 99: 659-667, (2008)].

A further method to increase the effective molecular weight of proteins and to produce a product which has reduced immunogenicity is to coat the protein in polyethylene glycol (PEG). The in-vivo half-life of GH has been increased by conjugating the GH with polyethylene glycol, which is termed “pegylation” [see Abuchowski et al., J.Biol Chem., 252:3582-3586 (1977)]. PEG is typically characterised as a non-immunogenic uncharged polymer. PEG is believed to slow renal clearance by providing increased hydrodynamic volume in pegylated proteins (Maxfield et al., Polymer, 16:505-509 (1975)). U.S. Pat. No. 5,849,535 also describe human GH (hGH) variants which are conjugated to one or more polyols.

A problem associated with pegylation of GH is that the affinity of pegylated GH for its receptor is reduced thereby requiring administration of high dosage regimes. This also applies to other pegylated proteins, for example granulocyte colony stimulating hormone [GCSF], the interferons and somatostatin. It would be desirable to reduce the clearance of protein biopharmaceuticals that does not result in adverse immune response or reduced biological activity.

GH is an anabolic cytokine hormone important for linear growth in childhood and normal body composition in adults. GH acts through a cell-surface type 1 cytokine receptor (GHR). In common with other cytokine receptors, the extracellular domain of the GHR is proteolytically cleaved and circulates as a binding protein (GHBP). Under physiological conditions GH is in part bound in the circulation in a 1:1 molar ratio by GHBP and this complex appears to be biologically inactive, protected from clearance and degradation. GH binds sequentially with two membrane bound growth hormone GHRs via two separate sites on GH referred as site 1 and site 2. Site 1 is a high affinity binding site and site 2 a low affinity site. A single GH molecule binds 1 GHR via site 1. A second GHR is then recruited via site 2 to form a GHR:GH:GHR complex. The complex is then internalised and activates a signal transduction cascade leading to changes in gene expression. The extracellular domain of GHR exists as two linked domains each of approximately 100 amino acids (SD-100), the C-terminal SD-100 domain being closest to the cell surface and the N-terminal SD-100 domain being furthest away. It is a conformational change in these two domains that occurs on hormone binding with the formation of the trimeric complex GHR-GH-GHR. Cytokine hormones like growth hormone have a short plasma half-life and require frequent administration. For example, GH replacement involves daily injections. In common with other cytokines, extracellular domain GH receptor circulates as a binding protein and naturally prolongs GH's biological half-life.

In WO2009/013461 we describe GH fusion proteins. These GH molecules are fused to an extracellular domain of GHR to form ligand/receptor fusions that form dimers. We herein describe fusion proteins that comprise the extracellular domain of GHR fused to a polypeptide that does not typically bind GHR [i.e. not growth hormone]. The properties conferred on these fusion proteins by GHR include altered pharmacokinetics and biological activity.

### Statements of Invention

According to an aspect of the invention there is provided a fusion polypeptide comprising the amino acid sequence of the extracellular binding domain of growth hormone receptor, or active binding part thereof, linked directly or indirectly, to a polypeptide comprising an amino acid sequence wherein said amino acid sequence is not growth hormone.

In a preferred embodiment of the invention said polypeptide comprises or consists of the extracellular binding domain of human growth hormone receptor.

In a preferred embodiment of the invention said polypeptide comprises or consists of the amino acid sequence as represented in Figure is (SEQ ID NO:1 or SEQ ID NO: 21).

In a preferred embodiment of the invention said polypeptide comprising the extracellular binding domain is modified by addition, deletion or substitution of at least one amino acid residue wherein said modified polypeptide substantially lacks growth hormone binding activity or has reduced growth hormone binding activity.

In a preferred embodiment of the invention the polypeptide is modified in the growth hormone binding domain of said extracellular binding domain.

In a preferred embodiment of the invention said modification is one or more of the amino acid sequences selected from the group consisting of: W169, R43, E44, 1103, W104, 1105, P106, 1164 and D165.

In a preferred embodiment of the invention said modification comprises or consists of deletion of amino acid residue tryptophan 104 of the amino acid sequence set forth in SEQ ID NO: 1 or SEQ ID NO: 21.

In a preferred embodiment of the invention said amino acid residue tryptophan 104 is substituted for one or more amino acid residues.

In a preferred embodiment of the invention tryptophan 104 is substituted for alanine as set forth in SEQ ID NO: 2 or SEQ ID NO: 22.

In an alternative preferred embodiment of the invention said modification comprises modification of amino acid residues 125-131 of the amino acid sequence as set forth in SEQ ID NO 1 or SEQ ID NO: 21.

Preferably, said modification is the deletion of all or part of amino acid residues 125-131 of the amino acid sequence set forth in SEQ ID NO 1 or SEQ ID NO: 21.

In a preferred embodiment of the invention the polypeptide is linked to the extracellular binding domain of growth hormone receptor and is positioned amino terminal to the receptor binding domain in said fusion polypeptide.

In an alternative preferred embodiment of the invention the polypeptide is linked to the extracellular binding domain of growth hormone receptor and is positioned carboxyl terminal to the receptor binding domain in said fusion polypeptide.

In an embodiment of the invention said fusion polypeptide comprising a native amino terminal signal peptide is replaced with a non-native amino terminal signal peptide.

The invention includes modifications to the fusion polypeptide that enhance translational efficiency which may optionally include the use of alternative amino terminal signal peptides. For example, cytokines typically will have an amino terminal signal peptide that is processed from a precursor polypeptide by sequence specific protease cleavage. In the non-limiting examples of the disclosure this could include the replacement of the GSCF or leptin amino terminal signal peptide with the amino terminal signal peptide of growth hormone.

In a preferred embodiment of the invention said fusion polypeptide is provided with or substitute for an amino terminal signal peptide comprising the amino acid sequence MATGSRTSLLLAFGLLCLPWLQEGSA [SEQ ID NO: 39].

In a preferred embodiment of the invention said polypeptide is linked to the receptor binding domain by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5, 6 or 7 copies of the peptide Gly Gly Gly Gly Ser.

In a still further alternative embodiment of the invention said fusion polypeptide does not comprise a peptide linking molecule and is a direct fusion of the polypeptide and the receptor binding domain.

In a further embodiment of the invention said fusion polypeptide comprises a cytokine.

Cytokines are involved in a number of diverse cellular functions. These include modulation of the immune system, regulation of energy metabolism and control of growth and development. Cytokines mediate their effects via receptors expressed at the cell surface on target cells. Cytokine receptors can be divided into three separate sub groups. Type 1 (growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif is also present in Type 2 (interferon family) and Type III (tumour necrosis factor family).

In a preferred embodiment of the invention said cytokine is selected from the group consisting of: leptin, erythropoietin, prolactin; tumour necrosis factor (TNF) granulocyte colony stimulating factor (GCSF), granulocyte macrophage colony stimulating factor (GMCSF), ciliary neurotrophic factor (CNTF), cardiotrophin-1 (CT-1), leukemia inhibitory factor (LIF) and oncostatin M (OSM).

In a preferred embodiment of the invention said fusion polypeptide comprises GCSF.

In a preferred embodiment of the invention of the invention said fusion polypeptide comprises the amino acid sequence set forth in SEQ ID NO: 3.

In a preferred embodiment of the invention said fusion polypeptide is provided with a non-native amino terminal signal peptide.

Preferably said non-native amino terminal signal peptide comprises the amino acid sequence MATGSRTSLLLAFGLLCLPWLQEGSA [SEQ ID NO: 39].

In a preferred embodiment of the invention said fusion polypeptide comprises the amino acid sequence set forth in SEQ ID NO: 9 or SEQ ID NO: 11.

In a preferred embodiment of the invention said fusion polypeptide comprises the amino acid sequence set forth in SEQ ID NO: 17 or SEQ ID NO: 18.

In a further preferred embodiment of the invention said fusion polypeptide comprises an amino acid sequence selected from the group consisting of: SEQ ID NO: 32, 34, 36 or 38.

In a preferred embodiment of the invention said fusion polypeptide is provided with a non-native amino terminal signal peptide.

Preferably said non-native amino terminal signal peptide comprises the amino acid sequence MATGSRTSLLLAFGLLCLPWLQEGSA [SEQ ID NO: 39].

In a preferred embodiment of the invention said fusion polypeptide comprises leptin.

In a preferred embodiment of the invention of the invention said fusion polypeptide comprises the amino acid sequence set forth in SEQ ID NO: 4.

In a preferred embodiment of the invention said fusion polypeptide comprises the amino acid sequence selected from the group: SEQ ID NO: 5 or SEQ ID NO: 7 or SEQ ID NO 19 or SEQ ID NO 20.

In a preferred embodiment of the invention said fusion polypeptide comprises the amino acid sequence selected from the group consisting of: SEQ ID NO: 24, 26, 28 or 30.

In a preferred embodiment of the invention said fusion polypeptide is provided with a non-native amino terminal signal peptide.

Preferably said non-native amino terminal signal peptide comprises the amino acid sequence MATGSRTSLLLAFGLLCLPWLQEGSA [SEQ ID NO: 39].

According to an aspect of the invention there is provided a nucleic acid molecule that encodes a fusion polypeptide according to the invention or a nucleic acid molecule that hybridizes to said nucleic acid molecule and encodes a polypeptide wherein said polypeptide has the biological activity associated with said polypeptide.

In a preferred embodiment of the invention there is provided a nucleic acid molecule that encodes a fusion polypeptide according to the invention.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90%, 95%, 96%, 97%, 98% or 99% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80%, Identity to Hybridize)
Hybridization: 5×-6×SSC at 65° C.−70° C. for 16-20 hours
Wash twice: 2×SSC at RT for 5-20 minutes each
Wash twice: 1×SSC at 55° C.−70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

In a preferred embodiment of the invention said cell is stably transfected. In an alternative preferred embodiment of the invention said cell is transiently transfected.

According to an aspect of the invention there is provided a fusion polypeptide according to the invention for use as a medicament.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents for example chemotherapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

An embodiment of the invention will now be described by example only and with reference to the following figures:

FIG. 1A is the amino acid sequence of human GHR extracellular domain (SEQ ID NO 21); and FIG. 1B (SEQ ID NO 22) is the human GHR extracellular domain modified at W104; signal peptides are shown in bold and are optional; FIG. 10 is GCSF (SEQ ID NO 3) and FIG. 1D is Leptin (SEQ ID NO 4); FIG. 1E (SEQ ID NO: 1) is the amino acid sequence of human GHR extracellular domain without signal sequence; and FIG. 1F (SEQ ID NO: 2) is the amino acid sequence of human GHR extracellular domain without signal sequence and modified at W104.

FIG. 2A provides a schematic of Leptin-link-GHBP (2N1).

FIG. 2B provides the amino acid sequence of 2N1 (SEQ ID NO: 5).

FIG. 2C provides the nucleotide sequence of 2N1 (SEQ ID NO: 6).

FIG. 3A provides a schematic of Leptin-link-GHBP-His (2N1-Hist).

FIG. 3B shows the amino acid sequence of 2N1-Hist (SEQ ID NO:7).

FIG. C shows the nucleotide sequence of 2N1-Hist (SEQ ID NO:8).

FIG. 4A is a western blot of transient expression of 2N1 and 2N1-Hist. 1. leptin (50 ng/lane), 2. leptin (10 ng/lane), 3. MW standards, 4. 2N1 (clone 1_1), 5. 2N1 (clone 1_3), 6. 2N1-Hist (clone 2_2), 7. negative control.

FIG. 4B is a western blot of expression of 2N1 from a stable CHO Flp In cell line. (An antibody that recognised leptin was used for both western blots).

FIG. 5: In vitro bioassay for 2N1—0.2 ml medium from cells transiently expressing 2N1 was used in a 1 ml reaction volume;

FIG. 6A provides a schematic of GCSF-link-GHBP.

FIG. 6B provides the amino acid sequence of GCSF-link-GHBP ([SEQ ID NO: 9) and the amino acid sequence of GCSF linked to GHBP without a signal sequence (SEQ ID NO: 17).

FIG. 6C provides the nucleotide sequence of GCSF-link-GHBP (SEQ ID NO: 10), with the signal sequence underlined.

FIG. 7A provides a schematic of GCSF-link-GHBP (W104A).

FIG. 7B provides the amino acid sequence of GCSF-link-GHBP (W104A) (SEQ ID NO: 11); the W104A mutation is shown in bold and underlined and the amino acid sequence of GCSF-link-GHBP (W104A) without the N-terminal sequence (SEQ ID NO: 18).

FIG. 7C provides the nucleotide sequence of GCSF-link-GHBP (W104A) (SEQ ID NO:12); the signal sequence is underlined, the W104A mutation is shown in bold and underlined.

FIG. 8A provides a western blot of transiently expressed GCSF-link-GHBP and GCSF-link-GCSF(W104A). 1. GCSF-link-GHBP #1, 2. GCSF-link-GHBP #2, 3. GCSF-link-GHBP(W104A) #1, 4. GCSF-link-GHBP(W104A) #2, 5. Positive control (GCSF-link-GCSFR) #1, 6. Positive control (GCSF-link-GCSFR) #2, 7. Negative control (media only) #1, 8. Negative control (media only) #2.

FIG. 8B provides a western blot of stably expressed GCSF-link-GHBP and GCSF-link-GCSF(W104A). 1. GCSF-link-GHBP from adherent cells, 2. GCSF-link-GHBP(W104A) from adherent cells, 3. GCSF-link-GHBP from suspension cells, 4. GCSF-link-GHBP(W104A) from suspension cells.

FIG. 9: Purification of GCSF-link-GCSF(W104A). The fusion protein was purified by IMAC using a Ni-resin column. The picture shows the SDS-PAGE gel of the relevant elution fractions from this purification;

FIG. 10: Purification of GCSF-link-GCSF(W104A). The fusion protein was purified by IMAC using a Ni-resin column. The picture shows the SDS-PAGE gel of the relevant elution fractions from this purification;

FIG. 11: Temperature stability of GCSF fusion polypeptide (non-reduced gel);

FIG. 12: Temperature stability (reduced gel); Lane 1: Untreated GCSF_GHBP, Lane 2: Room temperature, Lane 3: 4° C., Lane 4: −80° C. (Freeze/Thaw), Lane 5: Untreated GCSF_W104A_GHBP, Lane 6: Room temperature, Lane 7: 4° C., Lane 8: −80° C. (Freeze/Thaw);

FIG. 13: Extended stability study (non-reduced gel); Lane 1: Untreated GCSF_GHBP, Lane 2: Room temperature, Lane 3: 4° C., Lane 4: −80° C. (Freeze/Thaw), Lane 5: Untreated GCSF_W104A_GHBP, Lane 6: Room temperature, Lane 7: 4° C., Lane 8: −80° C. (Freeze/Thaw);

FIG. 14: AML-193 proliferation assay. AML 193 (peripheral blood; acute monocytic leukemia) cells proliferate when stimulated with IL-3, GM-CSF or GCSF;

FIG. 15 FACS analysis; % neutrophils in blood measured at 24h, 48h, 72h and 96h after injection of control, Filgrastim and 4F1;

FIG. 16 FACS analysis; % neutrophils in bone marrow (BM) measured at 24h, 48h, 72h and 96h after injection of control, Filgrastim and 4F1;

FIG. 17: FACS analysis; mobilisation of HPC to blood measured at 24h, 48h, 72h and 96h after injection of control, Filgrastim and 4F1;

FIG. 18 FACS analysis; mobilisation of HPC in BM measured at 24h, 48h, 72h and 96h after injection of control, Filgrastim and 4F1;

FIG. 19: PK study; concentration of Filgrastm and 4F1 measured in the serum over 50h;

FIG. 20: Coomassie stained 10% SDS-PAGE (non-reducing); Analysis of purified product 2N2 (from run#2), 1: Final purified product (˜5 microg per lane), 2: Broad range BioRad standards;

FIG. 21: Bioactivity of 2N2 analysed in dual luciferase reporter assay;

FIG. 22: Protein sequence of 2N2 comprising the W104A transition (SEQ ID NO 19);

FIG. 23: Protein sequence of 2N2 (SEQ ID NO: 20);

FIG. 24: nucleotide sequence of 2N3 (SEQ ID NO: 23);

FIG. 25: translated protein sequence of 2N3 (SEQ ID NO: 24);

FIG. 26: nucleotide sequence of 2N4 (SEQ ID NO 25);

FIG. 27: translated protein sequence of 2N4. (SEQ ID NO 26);

FIG. 28: nucleotide sequence of 2N5 (SEQ ID NO 27);

FIG. 29: translated protein sequence of 2N5 (SEQ ID NO 28);

FIG. 30: nucleotide sequence of 2N6 (SEQ ID No 29);

FIG. 31: translated protein sequence of 2N6. (SEQ ID NO 30);

FIG. 32: nucleotide sequence of 4F2 (SEQ ID NO 31);

FIG. 33: translated protein sequence of 4F2 (SEQ ID NO 32);

FIG. 34: nucleotide sequence of 4F2_W104A (SEQ ID NO: 33);

FIG. 35: translated amino acid sequence of 4F2_W104A (SEQ ID NO 34);

FIG. 36: nucleotide sequence of 4F3 (SEQ ID NO: 35);

FIG. 37: translated protein sequence of 4F3 (SEQ ID NO 36);

FIG. 38: nucleotide sequence of 4F3_W104A (SEQ ID NO 37);

FIG. 39: translated amino acid sequence of 4F3_W104A (SEQ ID NO 38)

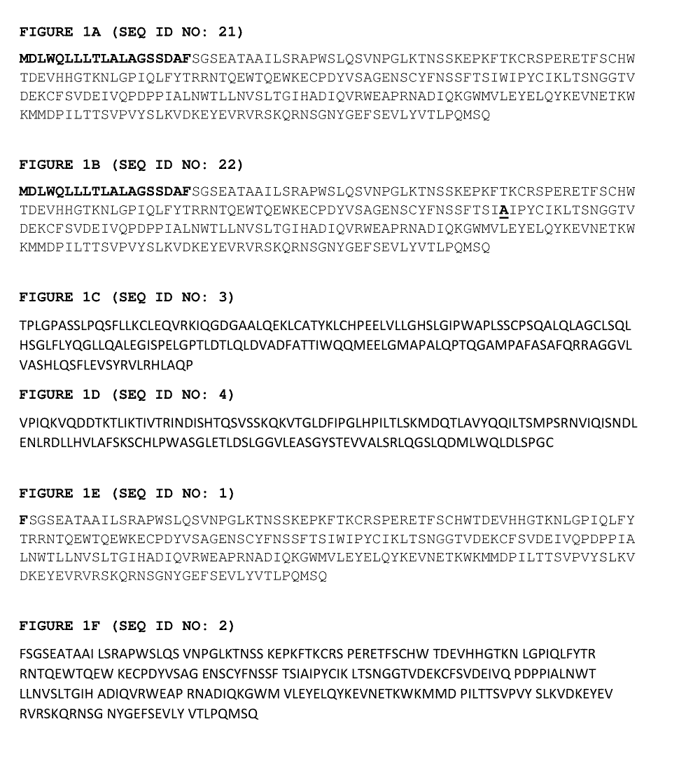

TABLE 1

| Full plasmid | | | |
|---|---|---|---|
| Name | | Description | Status |
| pObsecTag_2N1 | Ob-(G₄S)₄- | Leptin linked to GHBP | Constructed |
| | GHBP | via a (Gly₄Ser)₄ linker, | and |
| | | Contains leptin signal | crude media |
| | | sequence | tested: |
| pObsecTag_2N2 | Ob-(G₄S)₄- | As above but GHBP | Biologically |
| | GHBP | contains the W104A | active |
| | (W104A) | mutation | |

TABLE 2

| Full plasmid Name | Description |
|---|---|
| pOBsecTag_2N3 | leptin linked to GHBP (contains (G4S)5 linker) |
| pOBsecTag_2N4 | leptin linked to GHBP (contains (G4S)5 linker) |
| pOBsecTag_2N5 | leptin linked to GHBP (No linker) |
| pOBsecTag_2N6 | leptin linked to GHBP (No linker) |

TABLE 3

| Full plasmid Name | Description |
|---|---|
| pGCSFsecTag_4F2 | GCSF linked to GHBP (contains (G4S)5 |
| | linker) |
| pGCSFsecTag_4F2_W104 | GCSF linked to GHBP (contains (G4S)5 |
| | linker) |
| pOBsecTag_4F3 | GCSF linked to GHBP (No linker) |
| pOBsecTag_4F3_W104A | GCSF linked to GHBP (No linker) |

### Materials and Methods

### Generation of the Expression Plasmids

Both the leptin and GCSF fusion proteins were expressed from genes which had been constructed by using the polymerase chain reaction (PCR) to introduce NheI and NotI restriction sites at either end of the ligand gene and then inserting this into the gene for GH-link-GHBP to replace the GH sequence in this gene. The gene manipulations were carried out in the plasmid vector pGHSecTag1B8v0 (+/−His6-tag) (pSecTag/FRT/V5-His TOPO (Invitrogen) containing the gene for GH-link-GHBP); using the following primers.

LEPTIN PRIMERS
Forward primer
(SEQ ID 13)
5′-GGGAAAGCTAGCCACCATGCATTGGGGAACCCTGTG-3′
Reverse primer
(SEQ ID 14)
5′-ATTGATTAGCGGCCGCCGCACCCAGGGCTGAGGTCC-3′
GCSF PRIMERS
Forward primer
(SEQ ID 15)
5′-AAGCTGGCTAGCCACCATGGC-3′
Reverse primer
(SEQ ID 16)
5′-AATTAATAGCGGCCGCCGGGCTGGGCAAG-3′

The W104A mutation was introduced into the fusion protein by digestion, of a previously synthesised, GHBPW104A gene using AvrII and EcoRV, the fragment containing the mutation was then inserted into GHBP of the fusion protein sequences between the same restriction sites.

Plasmids were maintained in E. coli XL1 Blue cells and expression was carried out in mammalian Chinese Hamster Ovary (CHO) cells.

### Expression of the Fusion Proteins

Transient expression of the fusion proteins were carried out by using a transfection agent (Mirius or Fugene-6) to introduce the expression plasmid into CHO Flp-In cells (Invitrogen). CHO cells were first seeded into the wells of a 24-well culture plate at 2×105 cells/ml using 1 ml/well, the plate was incubated overnight at 37° C./5% CO₂ to allow the cells to attach. The following day 6 μl of Fugene-6 or Mirius was added to 100 μl serum free culture media and mixed; 4 μg plasmid DNA was then added [transfectant:DNA ratio of 3:2] and the solution mixed. After 15 minutes at room temperature the transfection mixture was added drop-wise into the individual wells containing the CHO cells. The cells were incubated overnight 37° C./5% CO₂ and the culture media replaced with serum free media. The media was harvested after 72 hours and analysed for expression of the protein of interest.

Stable expression of the fusion proteins was carried out in the same way as for transient expression; however after the overnight incubation post-transfection the media was replaced with media containing 600 μg/ml Hygromycin B. This selective pressure was maintained until most of the cells had died off and only the cells which had been stably transfected started to regrow. Once a stably expressing population of CHO cells had been obtained this was maintained using a lower concentration of Hygromycin B. The cells were then subsequently adapted to suspension growth in Hyclone SFM4CHO Utility culture medium and this suspension culture used to produce fusion protein for purification.

### Western Blot/Immunoblot

Protein samples were separated by SDS Polyacrylamide Gel Electrophoresis (SDS-PAGE) and then the protein on the gels transferred to PVDF or nitrocellulose membrane by electro-transfer. After blocking with 5% (w/v) milk powder the membrane was probed with a primary antibody which recognized the protein of interest (leptin or GCSF). The membrane was then washed and then a secondary HRP-conjugated antibody used which bound to the primary antibody. After another wash the membrane was exposed to ECL reagent and exposed to x-ray film in a dark room. This caused black bands to appear where the antibodies had sequentially bound at the location of the protein of interest.

### Detection/Quantification Using ELISA

A sandwich Enzyme Linked Immuno-Sorbant Assay (ELISA) was used to detect and quantify both leptin and GCSF fusion proteins. In the case of leptin; an anti-leptin capture antibody was bound to the wells of a 96-well plate, the plate was then blocked with 3% (w/v) bovine serum albumin, the protein samples (standard curve and unknowns) were added to the wells, and then another anti-leptin detection antibody added to the wells. A secondary HRP-conjugated antibody was then added to the wells and the amount of leptin measured using a colourmetric reagent which reacts to horse radish peroxidase (HRP). The colourmetric change is proportional to the amount of leptin in the protein samples. The GCSF fusion was measured in the same way but anti-GCSF capture and detection antibodies were used.

### Protein Purification

A CHO cell line stably expressing the protein of interest was seeded at a density of ˜0.5×106/ml and grown at 37° C./5% CO₂ in roller bottles with a maximum volume of 500 ml/bottle. Once the viability of the cells had dropped to ˜20-30% the culture media was harvested and concentrated approximately 10-fold. The concentrated culture media was diluted with an equal volume of 40 mM phosphate buffer, pH 7.4, 1M NaCl, 20% glycerol, 20 mM Imidazole and loaded onto a 5 ml Ni-chelate column which had been pre-equilibrated with 10 column volumes of 20 mM phosphate buffer, pH 7.4, 0.5M NaCl, 10% glycerol (Buffer A). The column was washed with 10 column volumes of Buffer A with 10 mM imidazole, followed 10 column volumes of 20 mM Na acetate buffer, 0.5 M NaCl, 10% glycerol, pH 6 (Buffer B). Bound protein was then eluted using Buffer B containing increasing concentrations of imidazole. All buffers were filtered prior to use and the sample was clarified by centrifugation prior to loading.

### Leptin Bioassay

The bioactivity of the leptin-link-GHBP fusion was measured using a dual-luciferase bioassay. Briefly, HEK293 cells were transfected with a plasmid expressing the leptin receptor, a plasmid expressing firefly luciferase under the control of SIE (reporter plasmid), a plasmid expressing Renilla luciferase under the control of a CMV promoter (control plasmid) and a plasmid expressing STAT3 (to increase endogenous STAT3 levels in the cells). These were grown in starvation media overnight and then stimulated with different concentrations of leptin or media from cells expressing the leptin-link-GHBP fusion; increasing levels of stimulation led to increased expression of firefly luciferase via the SIE reporter plasmid.

After 6 hours stimulation the cells were lysed and the levels of firefly and Renilla luciferase activities measured using the Dual-Luciferase Assay Kit (Promega) in a luminometer. The firefly luciferase signal was divided by the Renilla luciferase signal to correct for sample-to-sample variation and then this divided by the signal for an unstimulated sample to provide a fold induction over background.

### GCSF Bioassay

The bioactivity of GCSF-link-GHBP was measured using a cell proliferation assay. Briefly, AML-193 cells were washed with PBS and then resuspended in culture medium (no GM-CSF) at 5×105 cells/ml. 50 μl of the cells were pipetted into the wells of a 96 well-plate and then stimulated with 50 μl of a dose range of GCSF or GCSF-link-GHBP. The plate was then incubated at 37° C./5% CO2 for 3 days. 20 μl of CellTitre 96 AQueousNon-Radioactive Proliferation Assay Reagent (Promega) was then added to the wells. The colourmetric change caused by the CellTitre reagent was measured using a spectrophotometric plate reader; the OD measured was proportional to the number of cells in the well which in turn is proportional to the activity of GCSF or the fusion molecule.

### Protein Stability Studies

Stability studies were undertaken on protein constructs to look for degradation and formation of higher order structures over an 8 day period. Samples of each construct were kept at room temperature, 4 degrees Celsius (fridge) and −80 degrees Celsius (freeze/thaw). Analysis was undertaken using SDS PAGE and native PAGE followed by coomassie staining and western blotting.

### Extended Stability Study

To assess stability of protein samples over a longer period of time, 4C, RmT and −80C F/T samples were also analysed after 3 months incubation. Samples from this experiment were only analysed by SDS PAGE followed by coomassie staining.

### Animals

Specific pathogen-free (BDF1) male mice at (6-9) weeks of age are used for efficacy and pharmacokinetics studies. BDF1 mice were chosen for these studies because the pharmacokinetics of G-CSF are known to depend on the strain of mice used, and BDF1 mice have been shown to have a robust response to G-CSF (Haan et al 2000, Halpern et al 2002, Lord et al 2001, Molineux et al 1999). Mice are allowed to acclimate for 1 week prior to the start of the experiment. All animal handling and experimental procedures will be carried out under Home Office License according to the provisions of the United Kingdom Animals.

### Protein Preparations

Proteins to be tested and their concentrations are given in Table 1. 4F1-W104 samples are in PBS buffer.

TABLE 4
Proteins and their concentration

| | Concentration | | |
|---|---|---|---|
| Protein | (μg/ml) | Total Protein (mg) | Total Protein (nM) |
| Filgrastim | 300 | | |
| 4F1-W104 | 450 | 13.95 | 9595 nM |

The working concentrations of 4-F1-W104 for pharmacokinetic and pharmacodynamic studies are obtained by diluting as necessary in phosphate-buffered saline (PBS). The working concentrations of Filgrastim are obtained by diluting as necessary in 5% Dextrose.

### Protein Administration

Molar equivalence of the test proteins is based upon an average molecular weight of 46.9 kD for 4F1-W104 and reported MW of 19 kD for Filgrastim. Table 5 summarizes the moles (in nmol) for the dose administration used in these studies.

TABLE 5
Molar Equivalence

| | | Molar Equivalent |
|---|---|---|
| Protein | Dose administrated (mg/kg) | (nmol/kg) |
| Filgrastim | 0.25 | 13.150 |
| | 1.25 | 65.750 |
| 4F1-W104 | 0.25 | 5.33 |
| | 1.25 | 26.6 |

### Analysis the Samples for Pharmacodynamic Studies

As G-CSF is a haematopoietic cytokine that acts on neutrophil lineage cells and activates mature neutrophils, recombinant G-CSF has been widely used for adjuvant chemotherapy. The biological activity of the constructs can be evaluated by determining G-CSF activity of increasing WBC counts.

### Peripheral Blood Smear

Blood is collected from the tail vein into capillary tubes pre-treated with heparin and transferred into Capiject ethylene diamine tetra acetic acid (EDTA) tubes. For total white blood cell (WBC) count 10 μl of blood is added directly to 10 mL of Isoton II buffer containing four drops of Zapoglobin II for red blood cell (RBC) lysis. WBC count is determined via Coulter Counter.

The number of granulocytes and hematopoietic progenitor cells was assessed by flow cytometry using Gr-1 (granulocytes) and c-kit (hematopoietic progenitor cells) antibody markers. For staining, 20-50 μl of whole blood is added to an antibody cocktail containing FITC conjugated Gr.1/8C5, c-kit/CD117 conjugated to R-PE, and Mac-1/CD11b conjugated to PE-Cy5. Cells were incubated 20 min at room temperature.

Erythrocytes are lysed by a brief incubation with 0.5 mL ACK Lysing Buffer. The lysing reaction is stopped with 2 mL of FACS buffer, and the cells are pelleted by centrifugation. The cells are resuspended in FACS buffer (d-PBS with 0.1% sodium azide and 0.1% BSA) and acquired on the FACScan (Halpern et al 2002).

### Bone Marrow Biopsy

Bone marrow was prepared by extensive pipetting of bone marrow.

### Pharmacokinetics Studies

Mice are injected subcutaneously (S.C) in the mid-scapular region with 0.25 mg/kg of filgrastim, 4F1-W104, and PBS for vehicle control mice. Blood is collected via the inferior vena cava at 2, 6, 12, 24, 36, 48, 72 and 96 h after injection. Blood samples were centrifuged at 3000 rpm for 15 min (16000 g for 10 min) to obtain serum and then stored at −80° C. until analysis. The serum concentrations of G-CSF and 4F1-W104 were determined by human G-CSF ELISA kit in the presence of mouse serum.

Vehicle control mice received a single SC injection of PBS. Experimental mice received a single administration of Filgrastim and 4F1-W104 at a dose level of 0.25 mg/kg. The number of peripheral granulocytes and hematopoietic progenitor cells in mice is evaluated daily for 5 consecutive days (0, 24, 48, 72, 96h).

### Expression and Purification of 2N2

A stable CHO cell line was produced and protein expressed as a secreted product in roller bottle culture. Protein was purified from cell culture media using an anti-GHBP antibody column, dialysed in to PBS and concentrated.

### Example 1

### Leptin-link-GHBP (2N1)

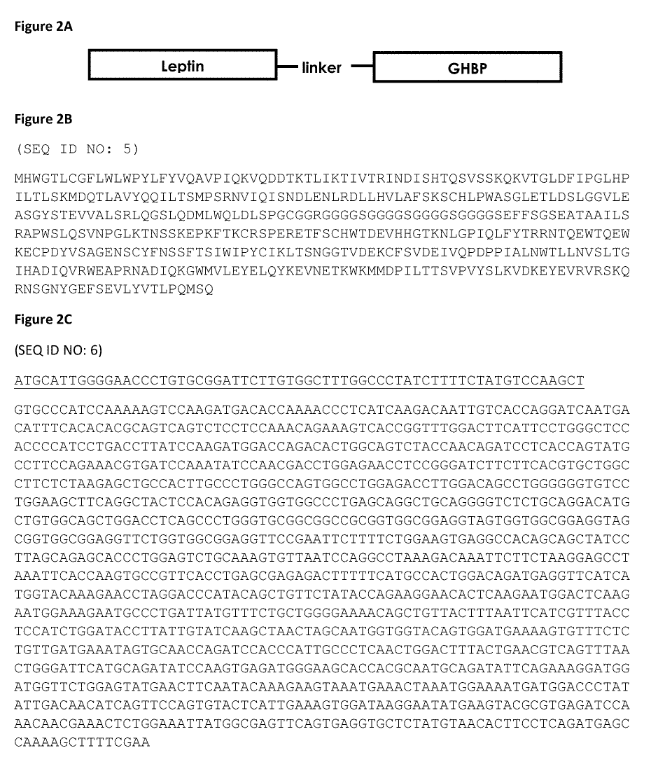

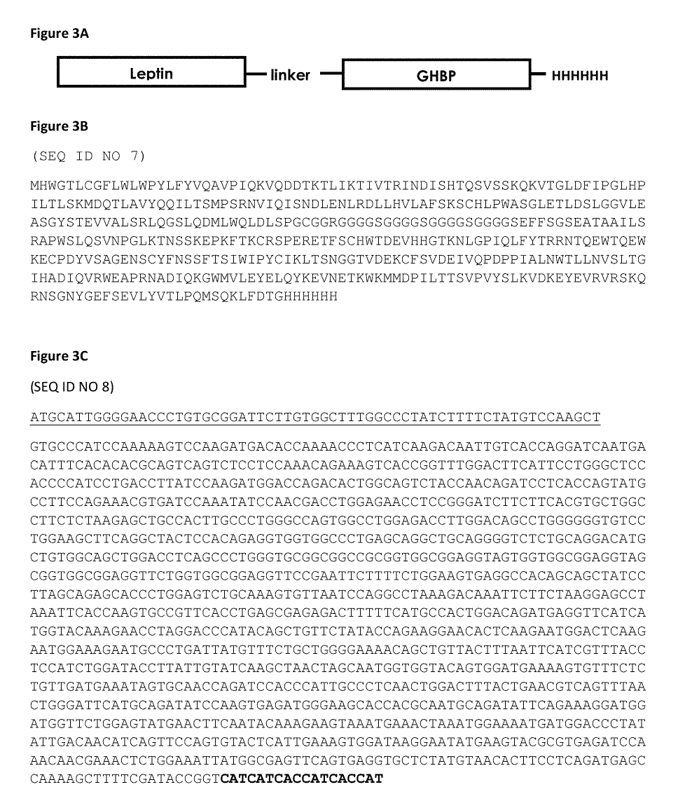

The protein fusion construct leptin-link-GHBP (2N1) was designed without (FIG. 2b; SEQ ID NO 5) and with a His×6 tag. The expression gene for these constructs were generated and inserted into the expression vector, pSecTag, using conventional molecular biology techniques e.g. PCR, restriction digestion and ligation.

Expression was first established as transient transfections in CHO Flpln cells, using Fugene-6 as the transfectant and a DNA:transfectant ratio of 2:3—the manufacturer's instructions were followed to achieve transfection. Expression was confirmed by western blot (FIG. 4A) using media from the adherent cell culture 72 hours post-transfection. The probe used for the western blot was anti-leptin antibody.

A stable cell line expressing 2N1 was then established by growing transfected CHO Flpln cells in the presence of Hygromycin B. The selective pressure of Hygromycin B ensured that only the CHO Flpln cells which had been stably transfected with the expression vector would survive. Expression from these cells was also confirmed by western blotting (FIG. 4B).

To demonstrate that the fusion protein had leptin-like bioactivity media from transiently expressing CHO Flpln cells was used in a leptin bioassay. Cells expressing the leptin receptor and containing a firefly luciferease reporter plasmid were stimulated with 0.2 ml media from cells transiently expressing 2N1. Stimulation of the leptin receptors initiated a signal cascade which resulted in the production of firefly luciferase from the reporter plasmid; production of the firefly luciferase was proportional to the stimulation of the leptin receptor. A constitutively expressing plasmid which produced Renilla luciferase was used to correct for inter-assay variability. Leptin was also used in the assay as a positive control and also to show a dose response. This dual-luciferase assay was measured using a luminometer, and the activity of leptin and 2N1 calculated as fold induction over the background luminosity (FIG. 5).

Thus, we show that leptin-link-GHBP can be produced and that this fusion protein has bioactivity.

### Example 2: Leptin-Link-GHBP (W104A in GHBP) (2N2)

Purified protein from 2 purification runs (Run #1 & Run#2) were analysed in the dual luciferase reporter assay. Both preparations show biological activity in the assay compared to PBS controls. A sample concentration of ˜450 nM was used in the assay. Both preparations show biological activity (FIG. 22).

### Example 3: GCSF-Link-GHBP

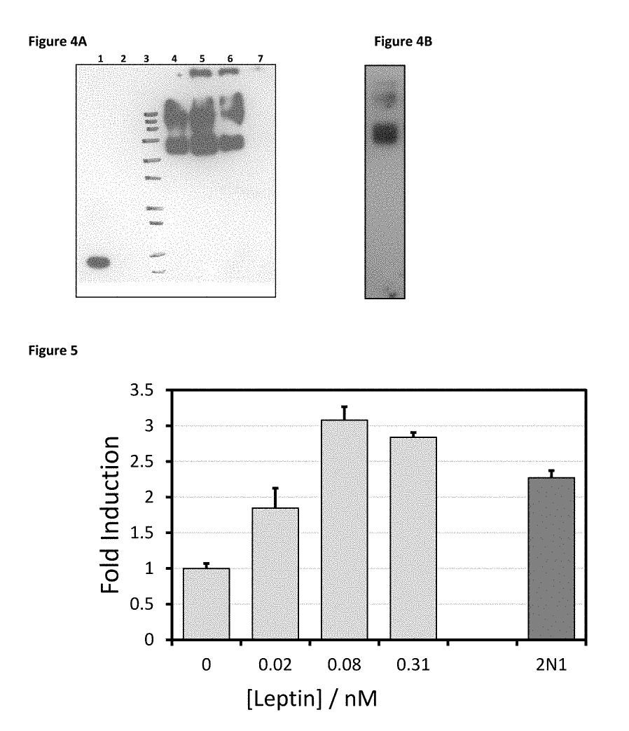

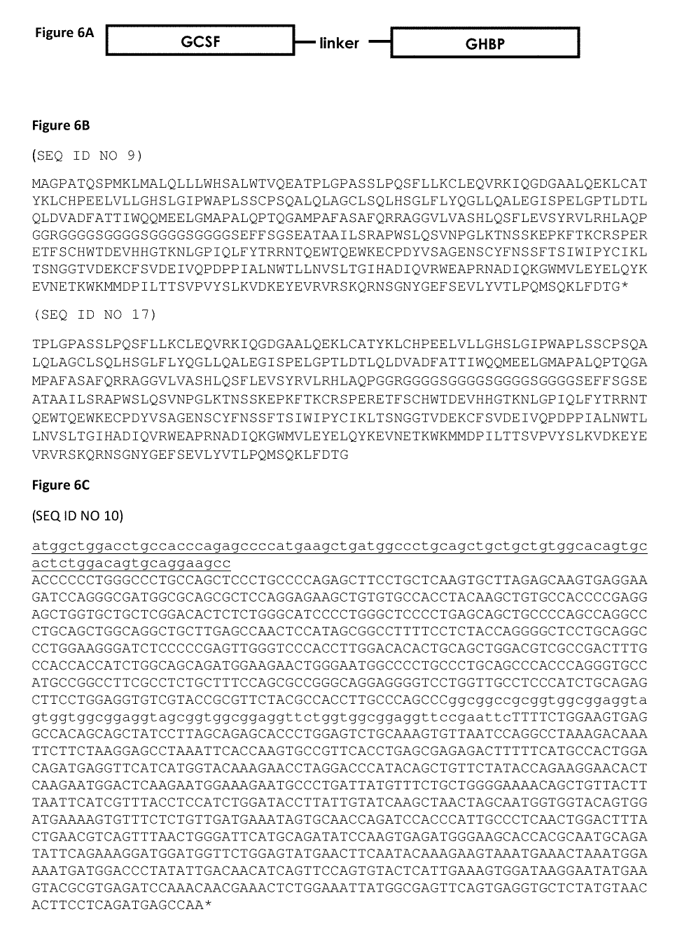

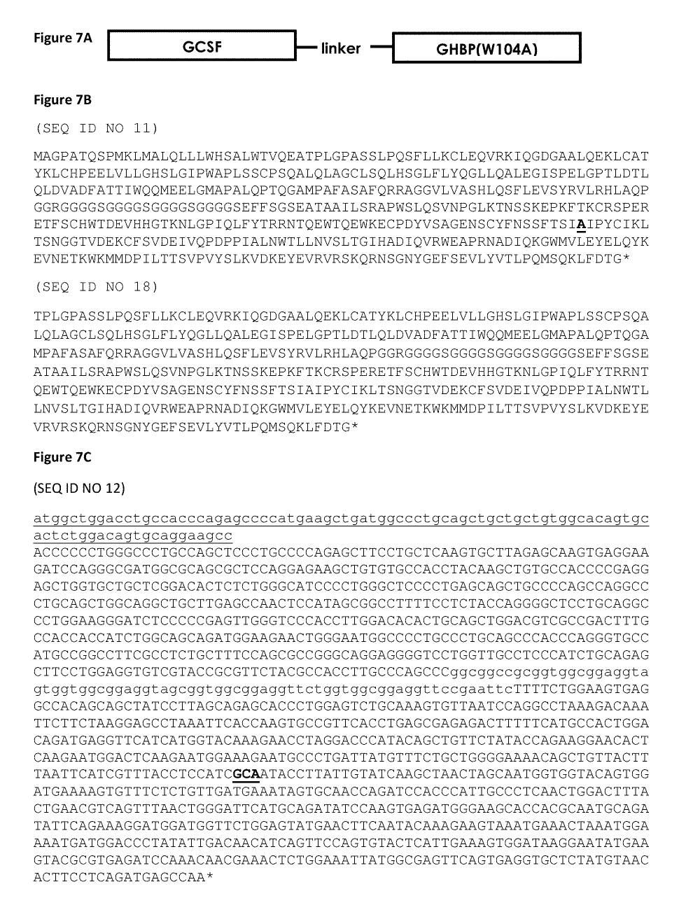

The protein fusion construct GCSF-link-GHBP was designed without (FIG. 6B; SEQ ID NO 9) and with a W104A mutation in GHBP (FIG. 7B, SEQ ID NO 11). The expression gene for these constructs were generated and inserted into the expression vector, pSecTag, using conventional molecular biology techniques e.g. PCR, restriction digestion and ligation.

Expression was first established as transient transfections in CHO Flpln cells, using Fugene-6 or Mirus transfection reagent as the transfectant and a DNA:transfectant ratio of 2:3—the manufacturer's instructions were followed to achieve transfection. Expression was confirmed by western blot (FIG. 8A) using media from the adherent cell culture 72 hours post-transfection. The probe used for the western blot was anti-GCSF antibody.

A stable cell line expressing GCSF-link-GHBP+/−W104A was then established by growing transfected CHO Flpln cells in the presence of Hygromycin B. The selective pressure of Hygromycin B ensured that only the CHO Flpln cells which had been stably transfected with the expression vector would survive. Expression from these cells was also confirmed by western blotting (FIG. 8B).

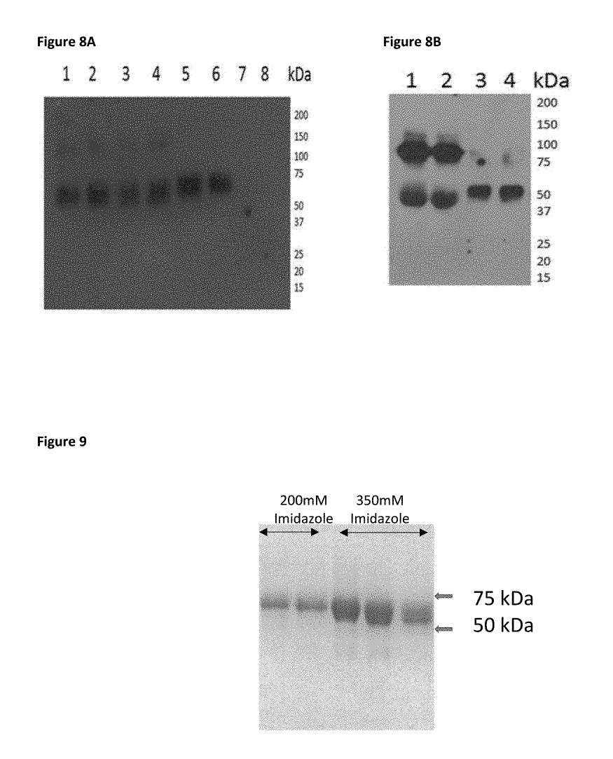

His-tagged GCSF-link-GHBP(W104A) was produced from a large volume of stably expressing CHO Flpln cells grown in suspension culture. The GCSF-link-GHBP(W104A) was then purified by immobilised metal ion chromatography (IMAC) using Ni-resin (FIG. 9).

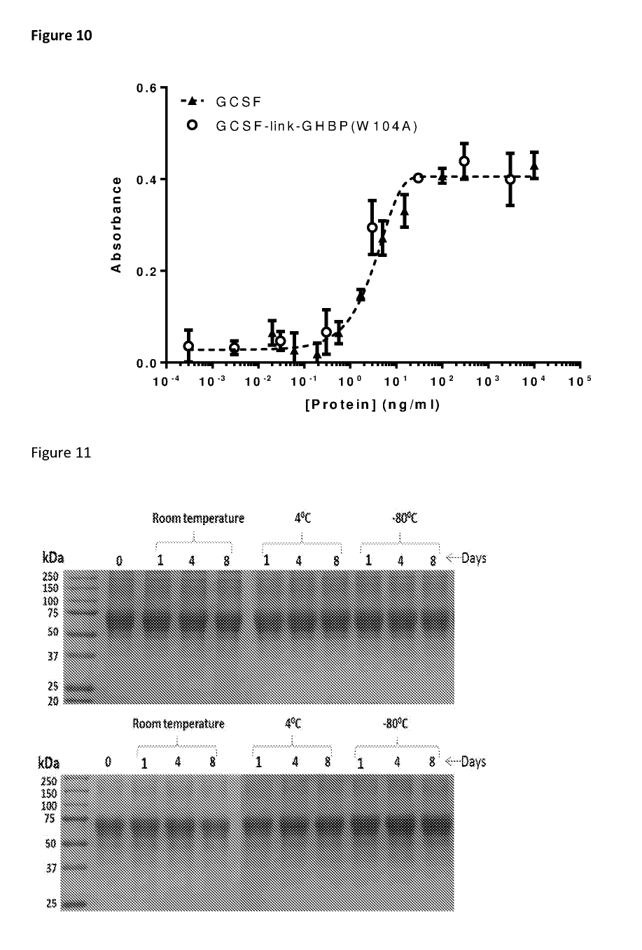

To demonstrate that the fusion protein had GCSF-like bioactivity a range of concentrations of the fusion protein were used to stimulate the growth of AML-193 cells. After 72 hours stimulation the proliferation of cells was measured using CellTitre reagent (Promega). The CellTitre reagent causes a colourmetric change proportional to the number of cells in the sample; the number of cells in the sample is in turn proportional to the activity of GCSF. A dose range of GCSF was also used as a positive control and also to compare the activity of the fusion protein. The activities of GCSF and GCSF-link-GHBP(W104A) were plotted as absorbance against protein concentration (FIG. 10) and the EC50s calculated; GCSF and GCSF-link-GHBP(W104A) have EC50s of 4 ng/ml (215 pM; Mw of 18.6 kDa) and 2 ng/ml (43 pM; assuming a Mw of 46.7 kDa), respectively. These calculations show that the fusion protein is approximately 5 times more active than native GCSF.

Thus, we show that GCSF-link-GHBP+/−W104A can be produced and that GCSF-link-GHBP(W104A) can be purified and has bioactivity comparable to that of GCSF.

### Example 4: Protein Stability Studies

A. Non-reduced gel: Test samples were incubated at room temperature, 4° C., and −80° C. (Freeze/Thaw). Samples were taken on day 0, 2, 4 and 8 and analysed by SDS-PAGE followed by Coomassie staining (5 μg protein loaded per lane). Both GCSF_GHBP (Top) and GCSF_W104A_GHBP (Bottom) showed no visible signs of degradation under all conditions studied over the 8 day period (FIG. 11).

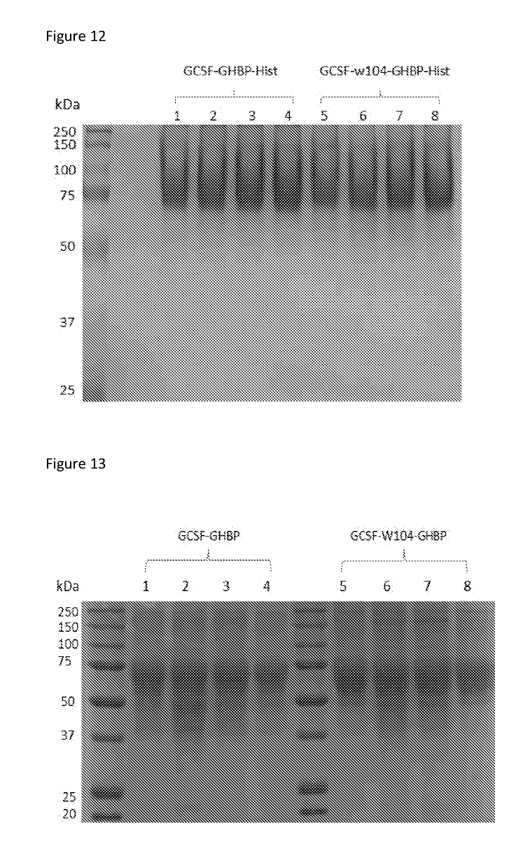

B. Reduced Gel: Test samples were incubated at room temperature, 4° C., and −80° C. (Freeze/Thaw). Samples were taken on day 8 and analysed by native PAGE followed by Coomassie staining (4 μg protein loaded per lane). No visible signs of degradation under all conditions (FIG. 12).

C. Non-reduced gel: Test samples were incubated at room temperature, 4° C., and −80° C. (Freeze/Thaw) for 3 months. Samples were analysed by SDS-PAGE followed by Coomassie staining (4 μg protein loaded per lane). No visible signs of degradation for −80° C. samples. Minimal degradation for room temperature, and 4° C. samples (FIG. 13).

### Example 5: In Vitro Bioactivity Evaluation of Proteins Using an AML-193 Cell-Based Proliferation Assay

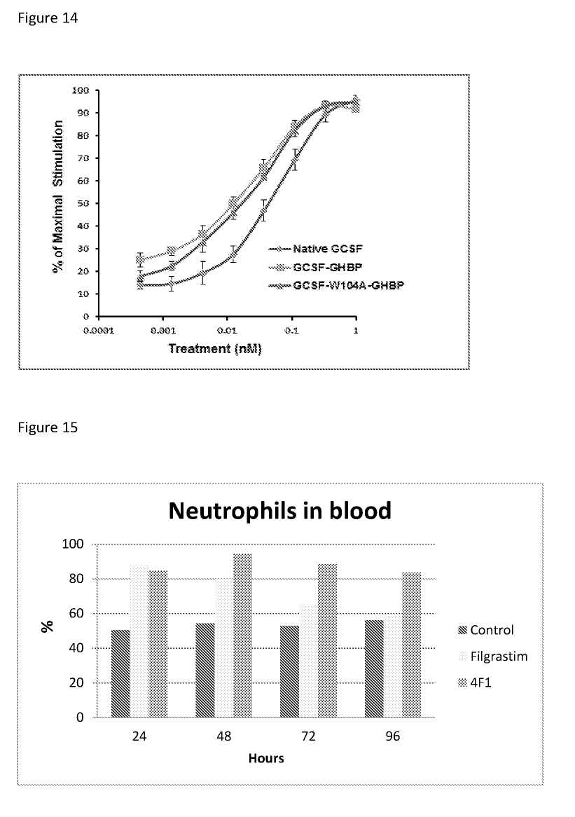

AML 193 (peripheral blood; acute monocytic leukemia) cells proliferate when stimulated with IL-3, GM-CSF or GCSF. Proliferation is dose dependent and can be used to evaluate the activity of the GCSF solutions. Both purified proteins showed increased bioactivity with all standard curves shifted to the left in comparison to native GCSF (FIG. 14, Table 6).

TABLE 6
EC50 values for each construct

| Construct | EC50 (nM) | SEM |
|---|---|---|
| Native GCSF | 0.056 (n = 12) | 0.009 |
| GCSF_GHBP | 0.024 (n = 6) | 0.004 |
| GCSF_W104A_GHBP | 0.020 (n = 6) | 0.004 |

### Example 6: In Vivo Analysis

### FACS Analysis

A: For Filgrastim group, the maximum mobilisation of peripheral neutrophils occurred on day 1 after injection, but on day 2 for 4F1 (FIG. 15).

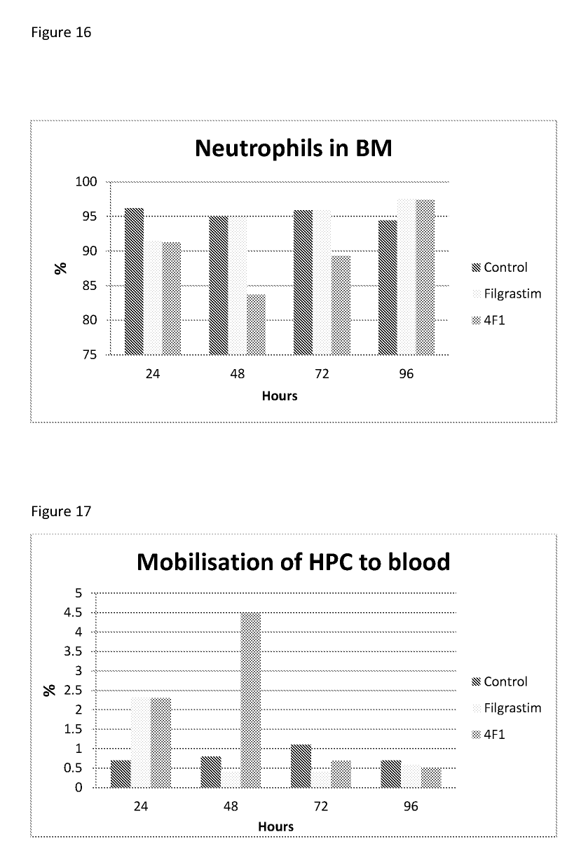

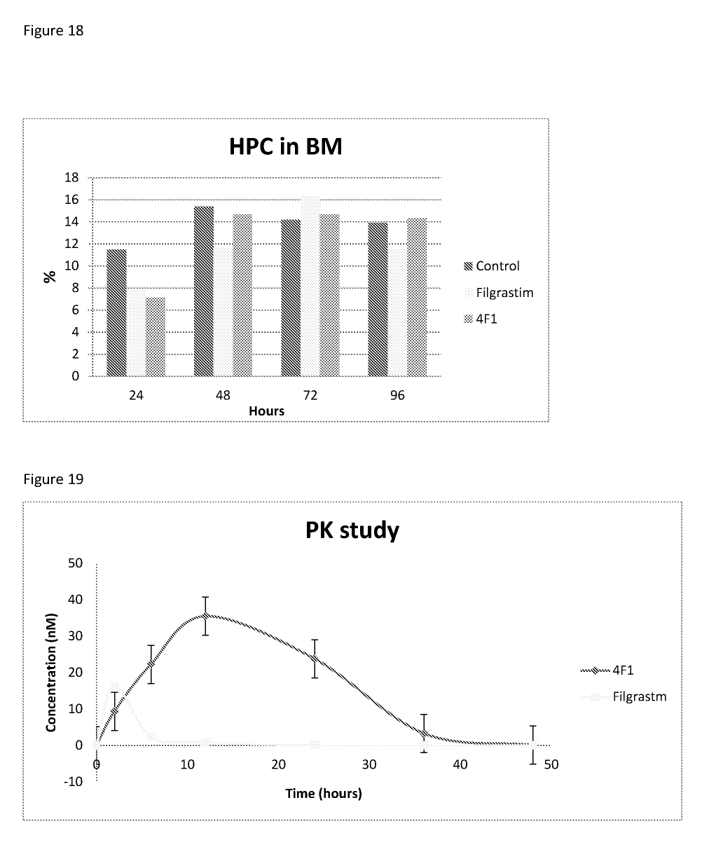

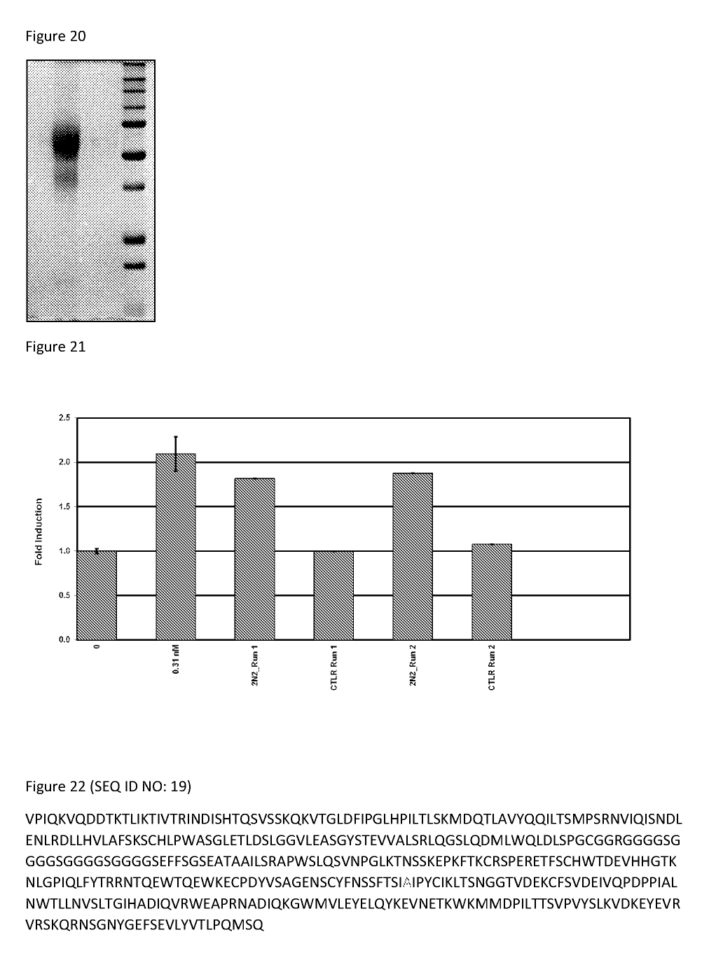

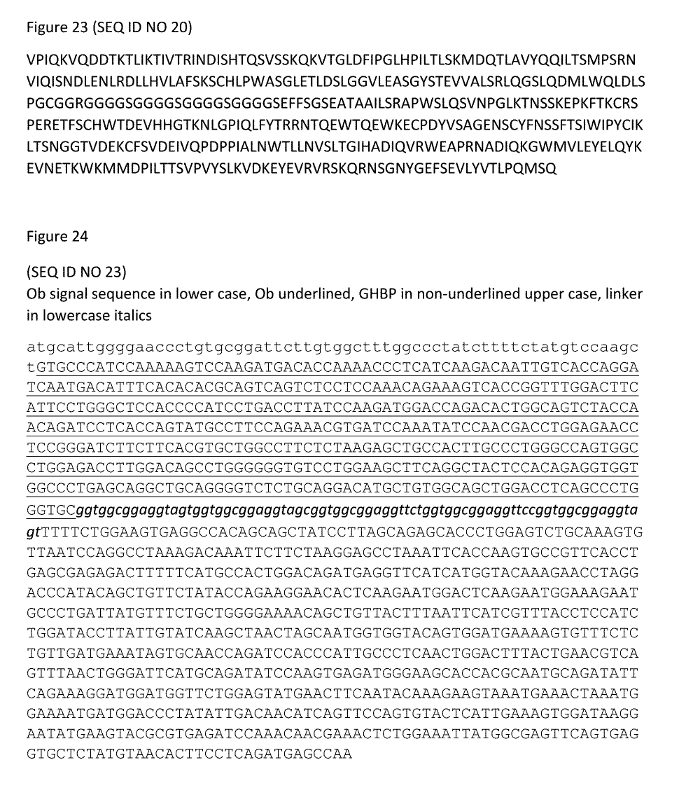

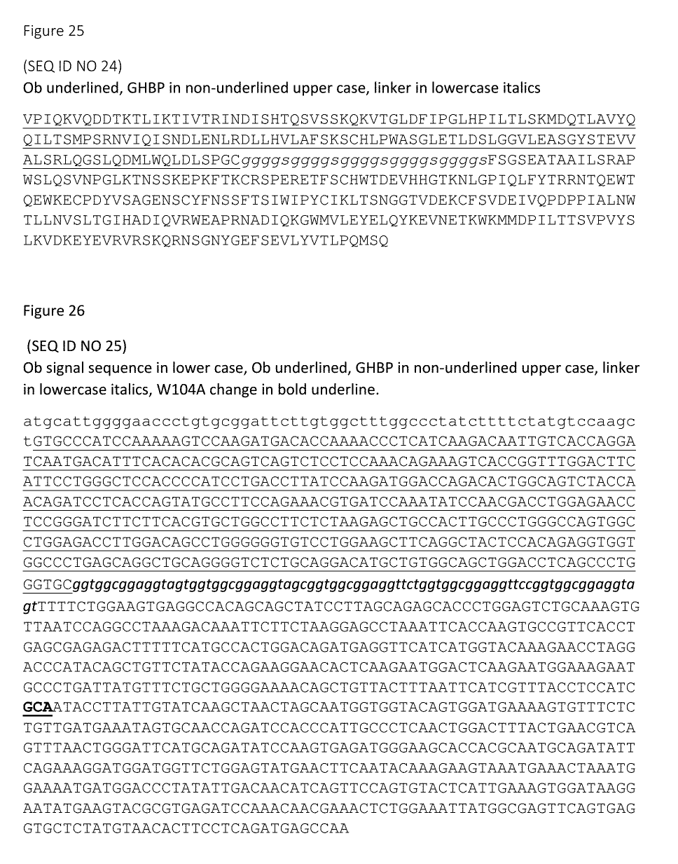

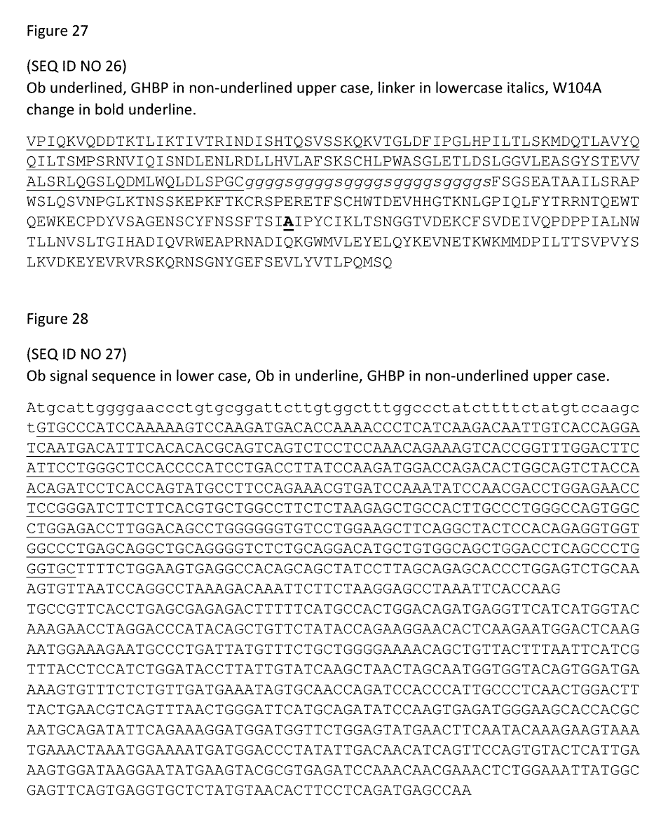

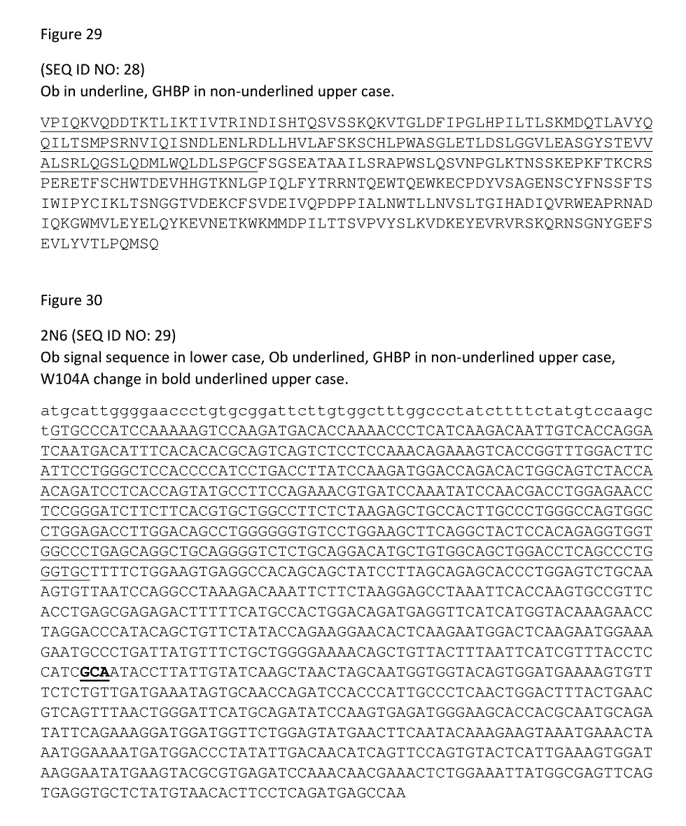

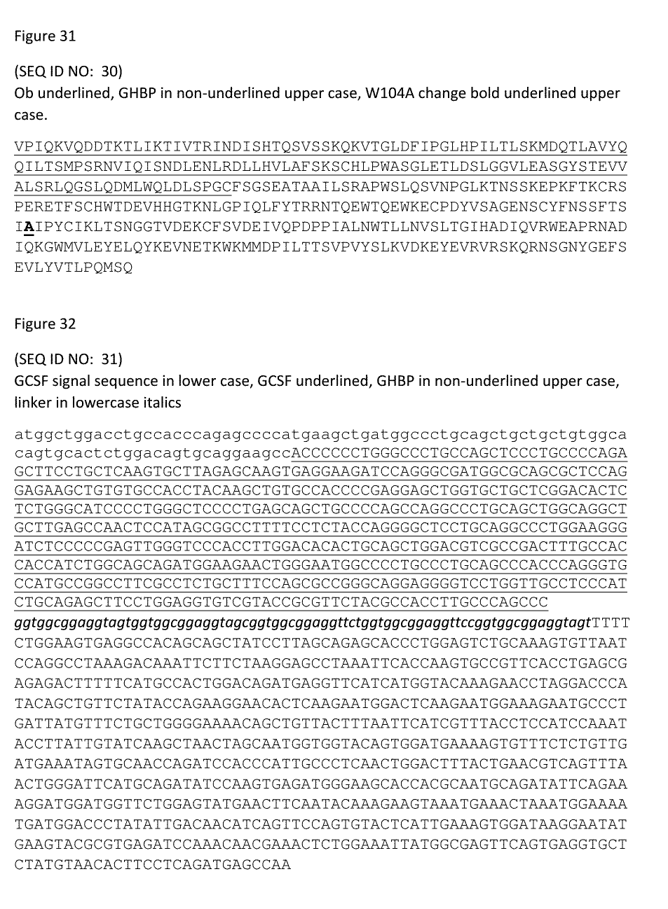

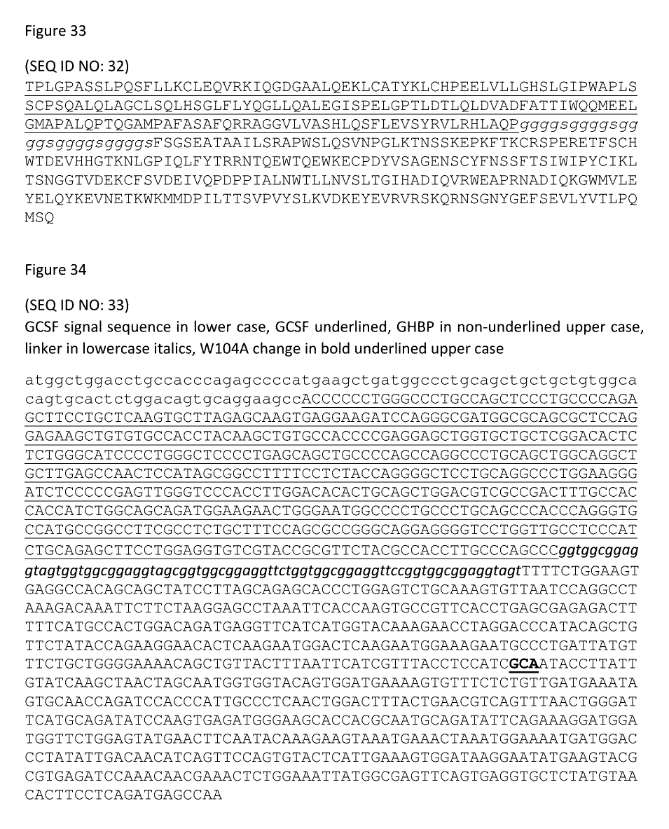

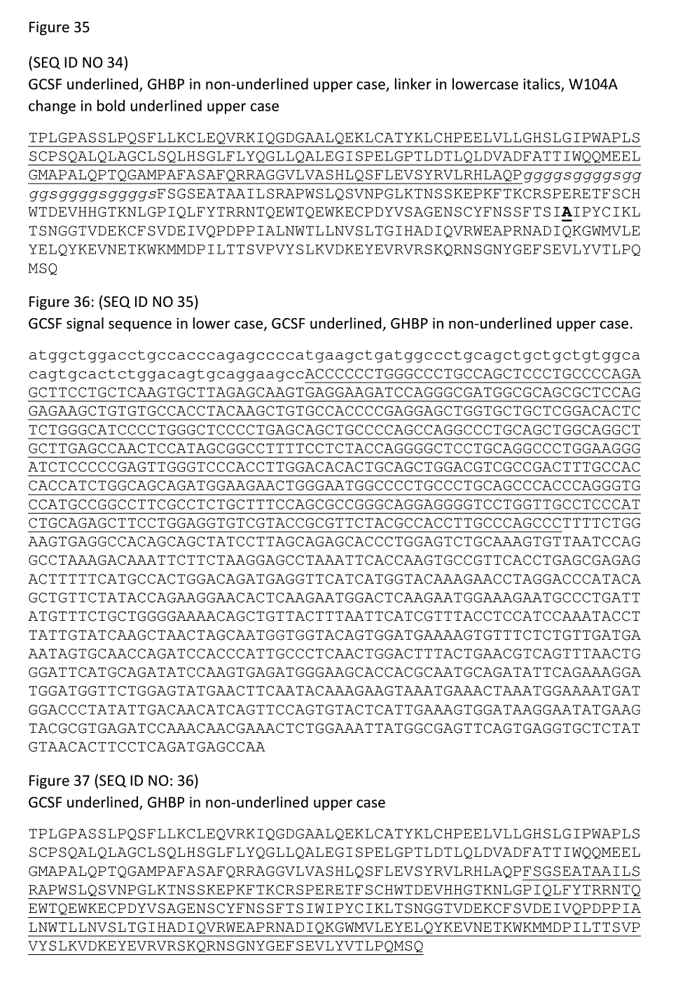

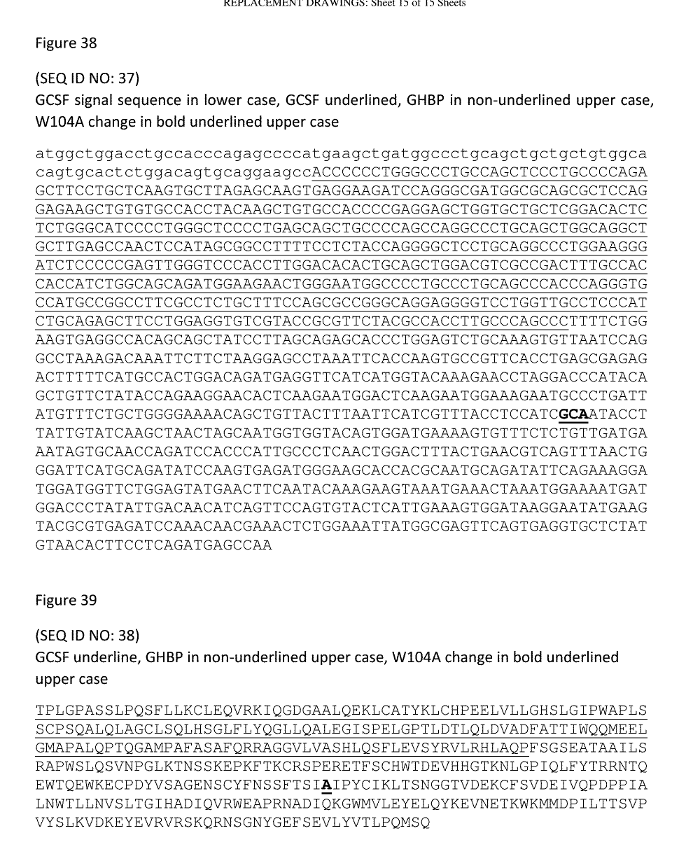

B: After a single administration of Filgrastim, the neutrophil count in the BM decreased at 24 hr, then returned to normal levels at 48 hrs. After a single administration of 4F1, the neutrophil count in the BM started to decrease at 24h. It reached their lowest count at 48 hrs. Then returned to normal levels at 96 hrs, (FIG. 16) The maximum mobilisation of HPC occurred on day 1 for Filgrastim, while on day 2 for 4F1 group. HPC returned to normal levels by day 2 for Filgrastim, while on day 3 for 4F1 group. (FIG. 17). After a single administration of Filgrastim and 4F1, HPC count in the BM decreased at 24 hrs, then started to return to normal levels by 96 hrs. (FIG. 18).

### PK Study

FIG. 19 shows that 4F1-W104 has a much better delayed clearance compared to Filgrastim, with a peak concentration around 12-24 hours and also shows that it might also have a slow rate of absorbance.

TABLE 7
Summary of SEQ ID NOs.

| | |
|---|---|
| SEQ ID NO: 1 | amino acid sequence of human GHR extracellular domain |
| SEQ ID NO: 2 | amino acid sequence of human GHR extracellular domain W104 mutation |
| SEQ ID NO: 3 | amino acid sequence of GCSF |
| SEQ ID NO: 4 | amino acid sequence of leptin |
| SEQ ID NO: 5 | amino acid sequence of leptin linked to GHBP |
| SEQ ID NO: 6 | nucleotide sequence of leptin linked to GHBP |
| SEQ ID NO: 7 | amino acid sequence of leptin linked to GHBP |
| SEQ ID NO: 8 | nucleotide sequence of leptin linked to GHBP with histidine tag |
| SEQ ID NO: 9 | amino acid sequence of GCSF linked to GHBP with signal sequence |
| SEQ ID NO: 10 | nucleotide sequence of GCSF linked to GHBP with signal sequence |
| SEQ ID NO: 11 | amino acid sequence of GCSF linked to GHBP with W104 mutation |
| SEQ ID NO: 12 | nucleotide sequence of GCSF linked to GHBP with W104 mutation |
| SEQ ID NO: 13 | leptin forward primer |
| SEQ ID NO: 14 | leptin reverse primer |
| SEQ ID NO: 15 | GCSF forward primer |
| SEQ ID NO: 16 | GCSF reverse primer |
| SEQ ID NO: 17 | amino acid sequence of GCSF linked to GHBP without signal sequence |
| SEQ ID NO: 18 | amino acid sequence of GCSF linked to GHBP with W104 mutation, |
| SEQ ID NO: 19 | 2N2 (W104) |
| SEQ ID NO: 20 | 2N2 |
| SEQ ID NO: 21 | amino acid sequence human GHR/ECD with signal sequence |
| SEQ ID NO: 22 | amino acid sequence human GHR/ECD with W104 and signal sequence |
| SEQ ID NO: 23 | nucleotide sequence of 2N3 construct (leptin fusion protein) |
| SEQ ID NO: 24 | amino acid sequence of 2N3 construct (leptin fusion protein) |
| SEQ ID NO: 25 | nucleotide sequence of 2N4 construct (leptin fusion protein) |
| SEQ ID NO: 26 | amino acid sequence of 2N4 construct (leptin fusion protein) |
| SEQ ID NO: 27 | nucleotide sequence of 2N5 construct (leptin fusion protein) |
| SEQ ID NO: 28 | amino acid sequence of 2N5 construct (leptin fusion protein) |
| SEQ ID NO: 29 | nucleotide sequence of 2N6 construct (leptin fusion protein) |
| SEQ ID NO: 30 | amino acid sequence of 2N6 construct (leptin fusion protein) |
| SEQ ID NO: 31 | nucleotide sequence of 4F2 construct (GCSF fusion protein) |
| SEQ ID NO: 32 | amino acid sequence of 4F2 construct (GCSF fusion protein) |
| SEQ ID NO: 33 | nucleotide sequence of 4F2 construct (GCSF fusion protein) W104 |
| SEQ ID NO: 34 | amino acid sequence of 4F2 construct (GCSF fusion protein) W104 |
| SEQ ID NO: 35 | nucleotide sequence of 4F3 construct (GCSF fusion protein) |
| SEQ ID NO: 36 | amino acid sequence of 4F3 construct (GCSF fusion protein) |
| SEQ ID NO: 37 | nucleotide sequence of 4F3 construct (GCSF fusion protein) W104 |
| SEQ ID NO: 38 | amino acid sequence of 4F3 construct (GCSF fusion protein) W104 |

1. A fusion polypeptide comprising:
the extracellular binding domain of growth hormone receptor, or active binding part thereof, linked directly or indirectly, to a polypeptide that is not the growth hormone.

2. (canceled)

3. The fusion polypeptide according to claim 1, wherein said extracellular binding domain of growth hormone receptor comprises or consists of the amino acid sequence of SEQ ID NO:1.

4. The fusion polypeptide according to claim 1, wherein said extracellular binding domain of growth hormone receptor, or active binding part thereof, is modified by addition, deletion or substitution of at least one amino acid residue, wherein said modified extracellular binding domain of growth hormone receptor, or active binding part thereof substantially lacks growth hormone binding activity or has reduced growth hormone binding activity.

5. The fusion polypeptide according to claim 4, wherein the extracellular binding domain of growth hormone receptor comprises a modification in the growth hormone binding domain.

6. The fusion polypeptide according to claim 4,5 wherein said modification is at one or more of amino acids: W169, R43, E44, I103, W104, 1105, P106, I164 and D165.

7. The fusion polypeptide according to claim 6, wherein said modification comprises or consists of deletion of W104 of SEQ ID NO: 1 or SEQ ID NO: 21.

8. The fusion polypeptide according to claim 6, wherein said W104 is substituted for one or more amino acid residues.

9. The fusion polypeptide according to claim 8, wherein W104 is substituted for alanine as shown in SEQ ID NO: 2 or SEQ ID NO: 22.

10. The fusion polypeptide according to claim 4, wherein said modification comprises modification of amino acid residues 125-131 of SEQ ID NO 1 or SEQ ID NO: 21.

11. The fusion polypeptide according to claim 10, wherein said modification is the deletion of all or part of amino acid residues 125-131 of SEQ ID NO 1 or SEQ ID NO: 22.

12. The fusion polypeptide according to claim 1, wherein the polypeptide that is not the growth hormone is linked to the extracellular binding domain of growth hormone receptor and is positioned amino terminal to a growth receptor binding domain in said fusion polypeptide.

13. The fusion polypeptide according to claim 1, wherein said polypeptide that is not the growth hormone is linked to the extracellular binding domain of growth hormone receptor and is positioned carboxyl terminal to a growth receptor binding domain in said fusion polypeptide.

14. The fusion polypeptide according to claim 1, wherein said polypeptide that is not the growth hormone is linked to the receptor binding domain by a peptide linker.

15. (canceled)

16. The fusion polypeptide according to claim 14, wherein said peptide linker comprises at least one copy of the peptide Gly Gly Gly Gly Ser.

17. The fusion polypeptide according to claim 16 wherein said peptide linker comprises 2, 3, 4, 5, 6 or 7 copies of the peptide Gly Gly Gly Gly Ser.

18. The fusion polypeptide according to claim 1, wherein said fusion polypeptide does not comprise a peptide linker and is a direct fusion of the polypeptide indirectly, to a polypeptide that is not the growth hormone and the extracellular binding domain of growth hormone receptor.

19. The fusion polypeptide according to claim 1, wherein said fusion polypeptide comprises a cytokine.

20. The fusion polypeptide according to claim 19 wherein said fusion polypeptide comprises GCSF.

21. The fusion polypeptide according to claim 20 wherein said fusion polypeptide comprises the amino acid sequence in SEQ ID NO: 3.

22. The fusion polypeptide according to claim 19, wherein said fusion polypeptide comprises the amino acid sequence of SEQ ID NO: 5, 7, 9, 11, 19, 20, 24, 26, 28, 30, 32, 34, 36 or 38.

23.-27. (canceled)

28. The fusion polypeptide according to claim 1, wherein said fusion polypeptide comprises a non-native amino terminal signal peptide.

29. The fusion polypeptide according to claim 28 wherein said non-native amino terminal signal peptide comprises the amino acid sequence MATGSRTSLLLAFGLLCLPWLQEGSA [SEQ ID NO: 39].

30. A nucleic acid molecule that encodes the fusion polypeptide of claim 1.

31. A vector comprising the nucleic acid molecule according to claim 30.

32. The vector according to claim 31 wherein said vector is an expression vector.

33. An isolated cell transfected or transformed with the nucleic acid molecule of claim 30.

34. A pharmaceutical composition comprising the fusion polypeptide of claim 1 and an excipient or carrier.

35. (canceled)
